(19) United States
(12) Patent Application Publication
(10) Pub. No.: US 2025/0322908 A1
(43) Pub. Date: Oct. 16, 2025

(54) METHODS AND SYSTEMS FOR RECOGNIZING AND QUANTIFYING GENETIC DEGENERACY AND FOR BIOMEDICAL APPLICATIONS OF GENETIC DEGENERACY QUANTIFICATIONS

(71) Applicant: NOBLIS, INC., Reston, VA (US)

(72) Inventors: Leo D. Thompson, Sterling, VA (US); Daniel Antonio Negrón, Annandale, VA (US); Jared Haas, Reston, VA (US); Justin Kyle Taylor, Leesburg, VA (US)

(73) Assignee: NOBLIS, INC., Reston, VA (US)

(21) Appl. No.: 18/631,794

(22) Filed: Apr. 10, 2024

(51) Int. Cl. G16B 30/00

(52) U.S. Cl. CPC G16B 30/00

(57) ABSTRACT

Methods for determining a genetic degeneracy score are described. The methods may comprise, for example, determining a window within the nucleotide sequence; determining one or more amino acids corresponding to one or more codons in the window; determining one or more degeneracy values for the one or more amino acids in the window; and combining the one or more degeneracy values in the window to determine the genetic degeneracy score. The methods may further comprise sliding the window by at least one nucleotide across the nucleotide sequence, to generate a plurality of genetic degeneracy scores. The methods may further comprise combining the plurality of genetic degeneracy scores into a final genetic degeneracy score.

### REFERENCE TO AN ELECTRONIC SEQUENCE LISTING

The contents of the electronic sequence listing (739642007400SEQLIST.xml; Size: 2,774 bytes; and Date of Creation: May 10, 2024) is herein incorporated by reference in its entirety.

### FIELD OF THE INVENTION

The present disclosure relates generally to methods and systems for analyzing genetic data, and more specifically to methods and systems for recognizing and quantifying genetic degeneracy using automated genetic degeneracy analysis systems, and for applying determined genetic degeneracy scores to biomedical, e.g., therapeutic, applications.

### BACKGROUND

Genetic codes underlie the composition of all known biological entities. That is, for all known biological entities, a nucleic acid sequence is transcribed and translated into an amino acid sequence. A given amino acid sequence, however, need not be the result of a single exclusive nucleic acid sequence. Oftentimes, multiple nucleic acid sequences can encode a given input amino acid sequence. Similarly, for a given input nucleic acid sequence, multiple other nucleic acid sequences can be determined, such that the determined and input nucleic acid sequences all have identical corresponding amino acid sequences. Such redundancy, may be referred to as genetic degeneracy.

### BRIEF SUMMARY OF THE INVENTION

As explained above, genetic degeneracy is an important feature of the genetic code. However, existing methods fail to efficiently and effectively leverage the genetic code's degeneracy for biotechnological, e.g., biomedical, applications. Specifically, known approaches do not provide for techniques to efficiently and effectively automatically recognize and quantify degeneracy of a nucleic acid sequence, nor for leveraging automatic recognitions and quantification (e.g., a degeneracy score) of degeneracy in biotechnological applications. Improved methods are needed for automatically recognizing and quantifying degeneracy of a nucleic acid sequence, and for automatically applying the recognized and quantified degeneracy quantification in various biotechnological applications. Disclosed herein are systems, methods, and techniques that may address the above identified needs.

Disclosed herein are methods and systems for determining a genetic degeneracy score, e.g., a genetic degeneracy score for a nucleic acid sequence. Existing methods for analyzing genetic sequences fail to properly consider the degenerate nature of the sequences. For example, mutations that do not alter the amino acid sequence, but alter the underlying nucleotide sequence—i.e., synonymous mutations—are under weak selection pressure. Thus, synonymous mutations occur relatively frequently across biological entities. Methods for designing probes, e.g., primers, against naturally occurring sequences often fail, because such methods do not accommodate for phenomena such as synonymous mutations, despite those mutations' relatively common occurrence. The methods and systems described herein address the shortcomings of the existing methods by providing a strategy for automatically recognizing and quantifying the degeneracy of a biological sequence, e.g., by determining genetic degeneracy scores. The genetic degeneracy scores can be incorporated in various biotechnological applications, such as automated pipelines for designing and producing primers against DNA sequences.

In some aspects, disclosed herein is a method of determining a genetic degeneracy score, comprising: receiving data comprising a representation of a nucleotide sequence, by one or more processors; determining a window within the nucleotide sequence, by the one or more processors; determining one or more amino acids corresponding to one or more codons in the window, by the one or more processors; determining, by the one or more processors, one or more degeneracy values for the one or more amino acids in the window; and combining the one or more degeneracy values in the window to determine the genetic degeneracy score, by the one or more processors.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A provides an exemplary method for determining a genetic degeneracy score.

FIG. 1B provides an exemplary method for determining a genetic degeneracy score, in the context of sequencing and processing a biological sample.

FIG. 1C provides an exemplary method for determining a genetic degeneracy score to determine a disease diagnosis for a subject.

FIG. 2 provides an exemplary schematic of a codon wheel.

FIG. 3 provides an exemplary table showing the number of codons that can encode a given amino acid.

FIG. 4 provides an exemplary flowchart for determining and using the genetic degeneracy score for a nucleotide sequence.

FIG. 5 depicts an exemplary computing device or system in accordance with one embodiment of the present disclosure.

FIG. 6 depicts an exemplary computer system or computer network, in accordance with some instances of the systems described herein.

FIG. 7 provides an example of a nucleotide sequence (SEQ ID NO: 1) for which genetic degeneracy scores are determined.

FIG. 8 provides an example depicting the determining of genetic degeneracy scores for the nucleotide sequence.

FIG. 9 provides an example plot of the determined genetic degeneracy scores, as depicted in FIG. 8.

FIG. 10 provides example data for determining genetic degeneracy scores for the nucleotide sequence depicted in FIG. 7.

FIG. 11 provides example data for visualizing genetic degeneracy scores based on the genetic degeneracy scores depicted in FIGS. 8 and 9.

### DETAILED DESCRIPTION

Methods and systems for determining genetic degeneracy scores are described herein. According to some embodiments of the disclosed methods, a nucleotide sequence is received, a window is determined within the nucleotide sequence, and amino acids corresponding to codons within the window are determined. Degeneracy values for the amino acids within the window are computed, and the degeneracy values in the window are then combined. A genetic degeneracy score for the window of the nucleotide sequence is generated based at least in part on the combined degeneracy values.

Naturally occurring biological entities, e.g., organisms and viruses, are functions of their underlying genetic sequence. Genetic sequences, however, are subject to mutations. Sequences that have unexpectedly mutated may fail to interact with designed biotechnological tools, such as probes, e.g., primers. For example, a primer may fail to hybridize against a nucleotide sequence, if the nucleotide sequence was subject to a mutation, such as a mutation on a nucleotide complementary to the 5′ end of the primer sequence. Most mutations, however, are detrimental enough to the fitness of the biological entity that the entity fails to propagate its genetic material to future generations, and the mutations are extinguished from the general population. That is, mutations detrimental to the entity's fitness are strongly selected against by natural selection.

In contrast, most mutations that survive across generations are not detrimental to the biological entity. Such mutations can either occur in non-coding regions of the entity's genome, or can occur in the coding regions, but are synonymous mutations, i.e., silent mutations. Synonymous mutations are mutations that alter a nucleotide sequence, but do not alter the corresponding amino acid sequence. Such mutations are possible, because oftentimes, an amino acid can be encoded by one of multiple possible nucleotide subsequences, e.g., codons. That is, nucleotide sequences are subject to a degenerate genetic code. For example, codons GGA, GGT, GGC, and GGG, can each encode the amino acid glycine. Accordingly, an example of a synonymous mutation is a mutation from the codon GGA to the codon GGT, i.e., the A mutates into a T. Such a mutation is a synonymous mutation, because even though the nucleotide sequence has changed from GGA to GGT, the corresponding amino acid sequence remains unaltered, from glycine to glycine (in this case, a sequence of a single amino acid).

Unlike non-synonymous mutations, e.g., mutations that are detrimental to entity fitness, synonymous mutations have limited fitness consequence on the entity. The limited fitness consequence comes from the fact that despite a change in nucleotide sequence, the output amino acid sequence is unaltered, and thus, the biological impacts that stem from the synonymous mutation are negligible. In addition, a fraction of non-synonymous mutations do not result in change in the biological entity's fitness. For example, some non-synonymous mutations result in an amino acid change in a non-essnetial region of a protein, such as, in the case of an enzyme, a non-active site. Additionally or alternatively, a non-synonymous mutation may result in a minimal effect on the secondary or tertiary structure of the protein. Such non-synonymous mutations and synonymous mutations can be considered to be neutral mutations or nearly neutral mutations. Given the limited fitness impacts of neutral and nearly mutations on a biological entity, neutral and nearly neutral mutations are hardly subject to selection pressure, and relative to other mutation types, are commonplace across populations of biological entities. Despite the ubiquity of neutral and nearly neutral mutations, existing methods of designing and configuring biotechnology tools often fail to accommodate or advantageously leverage neutral and non-neutral mutations. In general, biotechnology tools fail to capitalize on the genetic degeneracy of a nucleotide sequence. For example, primers are rarely designed with a target nucleotide sequence's degeneracy in mind. The methods disclosed herein address the shortcomings seen in existing methods.

When provided a nucleotide sequence, the methods disclosed herein perform an analysis within a selected window of the nucleotide sequence. The nucleotide sequence can be translated into an amino acid sequence, and a degeneracy value can be assigned to each amino acid in the amino acid sequence. The degeneracy values of the amino acids can be a function of the number of synonymous codons for each amino acid in the amino acid sequence. The degeneracy values within the applied window can be combined into a genetic degeneracy score for the portion of the nucleotide sequence demarcated by the window. The window can then be slid across the nucleotide sequence, and at each new window position (e.g., at each iteration) a new genetic degeneracy score can be calculated. The genetic degeneracy scores for all the window positions can be further combined and then normalized by nucleotide sequence length, to produce a final summary score.

The methods described herein benefit from being species agnostic. That is, the described methods for determining a genetic degeneracy score can be used for any biological entity, including prophetic, e.g., hypothetical, entities, provided that those entities are based on a genetic code. The methods described herein may therefore be of special relevance to, and may include practical applications in, the fields of bioengineering, e.g., synthetic biology, or biological defense, where artificial organisms based on engineered genetic codes may be used. Such artificial organisms can be analyzed, and tools, e.g., primers, targeting the artificial organisms can be effectively designed, based on the methods described herein.

Disclosed herein is a method of determining a genetic degeneracy score, comprising: receiving a nucleotide sequence, by one or more processors; determining a window within the nucleotide sequence, by the one or more processors; determining one or more amino acids corresponding to one or more codons in the window, by the one or more processors; determining, by the one or more processors, one or more degeneracy values for the one or more amino acids in the window; and combining the one or more degeneracy values in the window to determine the genetic degeneracy score, by the one or more processors.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The section headings used herein are for organizational purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature, and, as such, should not be viewed as limiting.

### Methods for Determining a Genetic Degeneracy Score

The disclosed methods for determining a genetic degeneracy score comprise: receiving a nucleotide sequence, by one or more processors; determining a window within the nucleotide sequence, by the one or more processors; determining one or more amino acids corresponding to one or more codons in the window, by the one or more processors; determining, by the one or more processors, one or more degeneracy values for the one or more amino acids in the window; and combining the one or more degeneracy values in the window to determine the genetic degeneracy score, by the one or more processors. The disclosed methods can further comprise sliding the window by at least one nucleotide across the nucleotide sequence, to generate a plurality of genetic degeneracy scores. The disclosed methods can further comprise combining the plurality of genetic degeneracy scores into a final genetic degeneracy score.

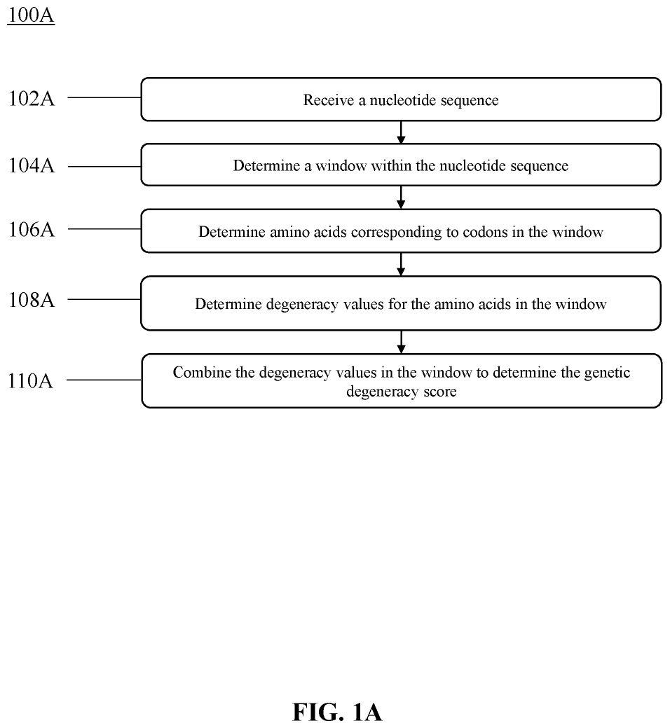

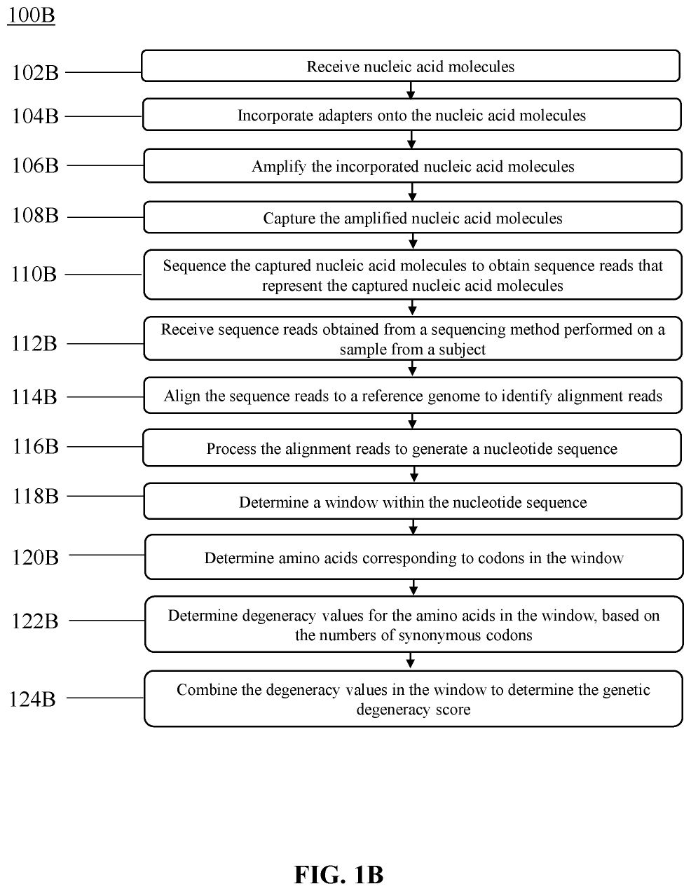

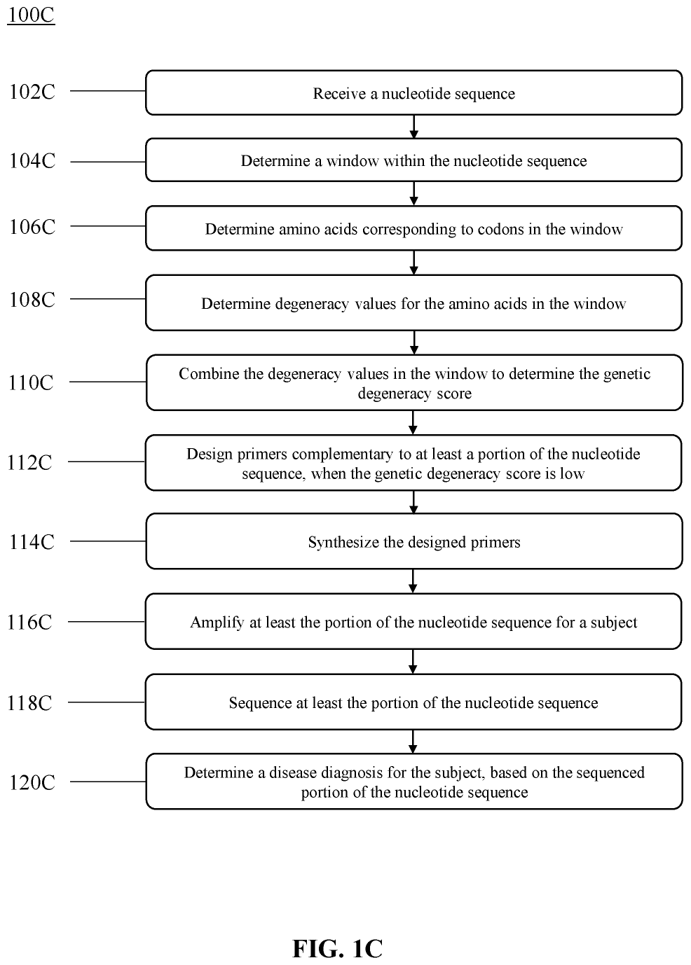

FIG. 1A shows an exemplary schematic showing a general process 100A for determining a genetic degeneracy score. The method can include: receiving a nucleotide sequence, by one or more processors (102A); determining a window within the nucleotide sequence, by the one or more processors (104A); determining one or more amino acids corresponding to one or more codons in the window, by the one or more processors (106A); determining, by the one or more processors, one or more degeneracy values for the one or more amino acids in the window (108A); and combining the one or more degeneracy values in the window to determine the genetic degeneracy score, by the one or more processors (110A).

FIG. 1B shows an additional exemplary schematic showing a general process 100B for determining a genetic degeneracy score for a sample from a subject. The method can include: receiving nucleic acid molecules obtained from the sample from the subject (102B); incorporating (e.g., ligating) one or more adapters onto one or more nucleic acid molecules from the nucleic acid molecules (104B); amplifying the one or more incorporated nucleic acid molecules from the nucleic acid molecules (106B); capturing the amplified nucleic acid molecules from the incorporated nucleic acid molecules (108B); sequencing, by a sequencer, the captured nucleic acid molecules to obtain sequence reads that represent the captured nucleic acid molecules (110B); receiving sequence reads obtained from a sequencing method performed on the sample from the subject, by one or more processors (112B); aligning the sequence reads to a reference genome to identify alignment reads, by the one or more processors (114B); processing the alignment reads to generate a nucleotide sequence, by the one or more processors (116B); determining a window within the nucleotide sequence, by the one or more processors (118B); determining one or more amino acids corresponding to one or more codons in the window, by the one or more processors (120B); determining, by the one or more processors, one or more degeneracy values for the one or more amino acids in the window (122B); and combining the one or more degeneracy values in the window to determine the genetic degeneracy score, by the one or more processors (124B).

FIG. 1C shows an additional exemplary schematic showing a general process 100C for determining a genetic degeneracy score for a sample from a subject. The method can include: receiving a nucleotide sequence, by one or more processors (102C); determining a window within the nucleotide sequence, by the one or more processors (104C); determining one or more amino acids corresponding to one or more codons in the window, by the one or more processors (106C); determining, by the one or more processors, one or more degeneracy values for the one or more amino acids in the window (108C); combining the one or more degeneracy values in the window to determine the genetic degeneracy score, by the one or more processors (110C); designing primers complementary to at least a portion of the nucleotide sequence, when the genetic degeneracy score is low (112C); synthesizing the designed primers (114C); amplifying at least the portion of the nucleotide sequence for a subject (116C); sequencing at least the portion of the nucleotide sequence (118C); and determining a disease diagnosis for the subject, based on the sequenced portion of the nucleotide sequence (120C).

Of note, step 104A of process 100A and step 104C of process 100C can be identical to step 118B of process 100B, wherein a window is determined within the nucleotide sequence, by the one or more processors; step 106A of process 100A and step 106C of process 100C can be identical to step 120B of process 100B, wherein one or more amino acids corresponding to one or more codons in the window are determined; step 108A of process 100A and step 108C of process 100C can be identical to step 122B of process 100B, wherein one or more degeneracy values for the one or more amino acids in the window are determined, by the one or more processors; and step 110A of process 100A and step 110C of process 100C can be identical to step 124B of process 100B, wherein the one or more degeneracy values are combined to determine the genetic degeneracy scores, by the one or more processors.

Process 100A, 100B or 100C can be performed, for example, using one or more electronic devices implementing a software platform. In some examples, process 100A, 100B or 100C is performed using a client-server system, and the blocks of process 100A, 100B or 100C are divided up in any manner between the server and a client device. In other examples, the blocks of process 100A, 100B or 100C are divided up between the server and multiple client devices. Thus, while portions of process 100A, 100B or 100C are described herein as being performed by particular devices of a client-server system, it will be appreciated that process 100A, 100B or 100C is not so limited. In other examples, process 100A, 100B or 100C is performed using only a client device or only multiple client devices. In process 100A, 100B or 100C, some blocks are, optionally, combined, the order of some blocks is, optionally, changed, and some blocks are, optionally, omitted. In some examples, additional steps may be performed in combination with the process 100A, 100B or 100C. Accordingly, the operations as illustrated (and described in greater detail below) are exemplary by nature and, as such, should not be viewed as limiting.

At 102A in FIG. 1A, a nucleotide sequence is received, by one or more processors. The nucleotide sequence can derive from a sample from a subject, e.g., the nucleotide sequence can be based on the sample from the subject. The subject, e.g., patient, can be a human.

Examples of the sample can include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample can be collected from the environment, i.e., the sample can be an environmental sample. The environmental sample can include a soil sample, a water sample, an air sample, or a combination thereof. The environmental sample can comprise biological entities, such as prokaryotic species or viruses. In some instances, the sample can include a combination of both a biological sample and an environmental sample.

The nucleotide sequence can be an engineered nucleotide sequence. That is, the nucleotide sequence may not occur in nature, and may instead, be synthesized under laboratory settings. The synthesis may involve chemical or enzymatic synthesis of the nucleotide sequence, or biotechnological synthesis of the nucleotide sequence, e.g., by cloning and/or splicing together fragments, as catalyzed by enzymes, such ligases or nucleases. Additionally, or alternatively, the nucleotide sequence can be a theoretical nucleotide sequence, e.g., a predicted nucleotide sequence. The theoretical nucleotide sequence can be a sequence that does not exist in nature, and may exist only in silico. The genetic code for the subject can be an artificial genetic code. An artificial genetic code can refer to any code that is not the naturally occurring genetic code. An artificial genetic code may comprise codon lengths that are not 3 nucleotides long, and codon lengths may even be variable for an artificial genetic code. The number of synonymous codons for a given amino acid in an artificial genetic code may differ from those of the naturally occurring genetic code. An artificial genetic code may not strictly comprise nucleotides that encode for amino acids, but may instead comprise some first sequence types that encodes some second sequence type, according to a set of rules. A genetic code, which can comprise a natural genetic code or an artificial genetic code, can include non-canonical amino acids, such as pyrrolysine or selenocysteine.

At 104A in FIG. 1A, a window within the nucleotide sequence is determined, by the one or more processors. The length of the window, in nucleotides, can be divisible by a length of a codon from the one or more codons, in nucleotides. Divisibility may refer to the dividing resulting in no remainder, i.e., if the length of the window is modulo the length of the codon, the result will be zero. The length of the window can be constant for a given nucleotide sequence. Similarly, the codon size for a codon can be constant for a given nucleotide sequence. The length of the window in nucleotides can be divisible by three. The length of the window in nucleotides can be at most equal to a length of the nucleotide sequence.

The end regions can be shorter in length than the length of the window. The end regions of the nucleotide sequence can be padded with padding values. Padding values may be necessary, if genetic degeneracy values are being determined for the first or last n−1 values of a nucleotide sequence (i.e., the end regions), and the window length is length n nucleotides. In which case, the number of genetic degeneracy values computed for the window containing the first n−1 values or earlier, or the last n−1 values or later, may be fewer than the number of genetic degeneracy values for other window positions. The first or last n−1 values may need to be padded with padding values. Alternatively, padding values may be necessary if a codon has length m nucleotides, in which case, an amino acid cannot be inferred for the first or last m−1 nucleotides of the nucleotide sequence. The first or last m−1 values may need to be padded with padding values. The padding values can comprise indeterminate values. Indeterminate values can be NaN values, as defined by IEEE-754 standards.

At 106A in FIG. 1A, one or more amino acids corresponding to one or more codons in the window are determined, by the one or more processors. The methods described herein can comprise using a codon look-up table for the determining the one or more amino acids corresponding to the one or more codons in the window. The codon look-up table can be a database of values for which a codon sequence and its corresponding amino acid are stored, for multiple codon sequences. The codon look-up table can be implemented computationally, e.g., as software. The one or more amino acids can include a stop signal, e.g., a signal encoded by a stop codon, which halts the further translation of nucleotides into amino acids.

At 108A in FIG. 1A, one or more degeneracy values for the one or more amino acids in the window are determined. The methods described herein can comprise using the codon look-up table for the determining the one or more degeneracy values. The codon look-up table can store the number of synonymous codons that encode an amino acid, for a plurality of amino acids. The codon look-up table can be a database of values relating to the number of synonymous codons that can encode an amino acid, for a plurality of amino acids. The codon look-up table can be implemented computationally, e.g., as software.

The one or more degeneracy values for the one or more amino acids in the window need not, for an amino acid, be the number of synonymous codons that can encode for the amino acid. The number of synonymous codons for an amino acid can be inputted into an arbitrary function to output a degeneracy value for the amino acid and/or codon. The arbitrary function can comprise computational aspects, such as transforming a computational object type into another object type, e.g., transforming an integer type value into a floating point type value.

A number of determined degeneracy values is fewer than the length of the window in nucleotides divided by the length of the codon. One degeneracy value from the one or more degeneracy values can be determined for each codon in the one or more codons. A degeneracy value from the one or more degeneracy values can range between 1 and 216. This range can be relevant to the naturally occurring genetic code. The degeneracy value can be an irrational number, a rational number, an integer, a whole number, or a natural number. The degeneracy value can be a computed integer or a computed float. A computed integer need not be the same as an integer, as used in mathematics, which can refer to a whole number (not a fractional number) that can be positive, negative, or zero. A computed integer can refer to an integer as used in computer science, which can refer to a datum of integral data type. A computed integer can differ from a computed float, in that the amount of memory allotted to a computed integer may be different, e.g., smaller, than the amount of memory allotted to a computed float.

At 110A in FIG. 1A, the one or more degeneracy values in the window are combined to determine the genetic degeneracy score for the window, by the one or more processors. The combining can comprise multiplying together the one or more degeneracy values in the window. The combining can comprise adding together the one or more degeneracy values in the window. Alternatively, the combining can be done with any arbitrary function that accepts as arguments, the one or more degeneracy values, and returns the genetic degeneracy score. For example, different degeneracy values within a window may be weighted by a scalar value, according to biological conditions, such as if the degeneracy values are related to a certain class of amino acids, e.g., by amino acid charge, or amino acid size.

The methods described herein can be iterated across multiple iterations. That is, the method can be iterative with respect to the determining the window, the determining the one or more amino acids, the determining the one or more degeneracy values, or the combining the one or more degeneracy values. The iterative method can stop after a predetermined number of iterations. The iterative method can stop after the sliding the window comprises sliding across the nucleotide sequence in its entirety. A first length of the window for a first iteration of the method can overlap with a second length of the window for a second iteration of the method. The methods described herein can further comprise sliding the window by at least one nucleotide across the nucleotide sequence, to generate a plurality of genetic degeneracy scores. The method can be iterative with respect to the sliding the window. The window length can be determined based on the method being applied to other nucleotide sequences. For example, a second nucleotide sequence of the same or similar biological entity or species as that of a first nucleotide sequence may have been analyzed using an window length of 15 nucleotides. Accordingly, the window length of 15 nucleotides can be used for analyzing the biological entity or species of the first nucleotide sequence. The window length can also be determined adaptively. That is, the method can be performed across a plurality of runs on the nucleotide sequence. During a first run of the plurality of runs, the window length can be set to a window length, e.g., a random window length, and with each iteration of the first run, the window can slide across the nucleotide sequence, from which one or more degeneracy scores can be determined and recorded and/or stored. During a second run of the plurality of runs, the window length be set to the random window length used during the first run, plus or minus some step size. For example, if the random window length during the first run was 15 nucleotides, the step size can be 2 nucleotides, and accordingly, the window length for the second run can be 13 nucleotides (or 17 nucleotides, if the step size is being added to the first run's window length, as opposed to subtracted). During the second run, the window length of size 13 nucleotides can, with each iteration of the second run, slide across the nucleotide sequence, from which one or more degeneracy scores can be determined and recorded and/or stored. The number of runs, where each run consists of a different window length, e.g., the window length of the previous run plus or minus some step size, can be iterated until a cessation condition is met. The cessation condition can be at least a minimum or a maximum degeneracy score. The step size need not be a constant step size, e.g., a step size of 2. Based on, for example, the changing degeneracy scores of the previous runs, the step size can increase or decrease to adjust the window length more dramatically or more finely.

The method can further comprise combining the plurality of genetic degeneracy scores into a final genetic degeneracy score. That is, for a nucleotide sequence, a single score, e.g., the final genetic degeneracy score, can be determined for the nucleotide sequence. The final genetic degeneracy score can be determined based at least in part on the plurality of genetic degeneracy scores that arise from sliding the window across the nucleotide sequence. For example, if the plurality of genetic degeneracy scores is a list of numbers like [1, 4, 16, 216, 18], determining the final genetic degeneracy score may involve combining the elements of the plurality of genetic degeneracy scores, e.g., by multiplying all the elements together. In order to accommodate for the fact that a larger final genetic degeneracy score may arise from a longer sequence, e.g., a larger plurality of genetic degeneracy scores, the final genetic degeneracy score can be normalized by the length of the sequence. For example, if the plurality of genetic degeneracy scores is the list of numbers [1, 4, 16, 216, 18], the product of the elements can be determined, i.e., 1*4*16*216*18=248832, and the product can be normalized by the length of the plurality of genetic degeneracy scores, e.g., 248832/5=49766.4. If desired, the value can be rounded to the nearest whole number to determine the final genetic degeneracy score, e.g., 49766.4 can be rounded to 49766. The rounded value can be represented computationally not as a float, but, for example, as an integer value. Alternatively, the final genetic degeneracy score can be determined, not by normalizing the product of the plurality of genetic degeneracy scores, but by performing an alternative operation, such as a logarithm. For example, if the plurality of genetic degeneracy scores is the list of number [1, 4, 16, 216, 18], then the product of the elements can be determined, i.e., 1*4*16*216*18=248832, and the product can be subjected to a base e logarithm: In 248832=12.42. The final degeneracy score need not be the result of multiple window positions. For example, the window can cover the entire nucleic acid sequence, in which case there may only be a single window position for the nucleotide sequence, provided that the bounds of the window do not exceed the bounds of the nucleotide sequence. The genetic degeneracy score can be based on the single window position. In such a case, the genetic degeneracy score for the nucleotide sequence can be equivalent to the final genetic degeneracy score. The genetic degeneracy score can also be calculated for a circular nucleotide sequence. The circular nucleotide sequence can be received as a linear nucleotide sequence.

To consider another open reading frame ƒ of s, s′ can be expressed as:

$${s}^{′} = {( {s}_{j} )}_{j = {f}^{′}}^{\vert s \vert} f ∈ \{ 1 , 2 , 3 \}$$

To consider the negative, i.e., antisense, strand of the nucleotide sequence, s′ can be expressed as:

$${s}^{′} = ( c ( {( {s}_{j} )}_{j = \vert S \vert - i + 1} ) \vert 1 \leq i \leq \vert s \vert )$$

where function c computes the complement, such as:

$$c ( x ) = \{ \begin{matrix}T , & x = A \\ G , & x = C \\ C , & x = G \\ A , & x = T\end{matrix}$$

The genetic degeneracy score can be combined with a determining a molecular clock rate for the nucleotide sequence. The molecular clock rate can refer to a theoretical, e.g., assumed, rate at which nucleotide sequences and/or proteins sequences of a biological species evolve over time, and the rate can be assumed to be constant. The molecular clock rate can be specific to a particular biological species, and the rate can vary across biological species. The molecular clock can be used to estimate the evolutionary time since a species diverged from another species. The molecular clock for a given species, as well as the genetic degeneracy score determined for a nucleotide sequence, can be combined, to generate, for example, probabilistic estimates of how likely synonymous mutations may occur for a given nucleotide in the nucleotide sequence.

The genetic degeneracy score can be used for a number of biotechnological, e.g., biomedical, applications. For example, the genetic degeneracy score can be used for identifying a conserved genetic sequence from a sample from a subject. That is, sequences with very low genetic degeneracy scores may be used to, in part, identify a conserved genetic sequence. The identified conserved genetic sequence can be used for detecting a pathogen. The pathogen can be an engineered pathogen. That is, the pathogen may be engineered, at least in part, under laboratory conditions. The engineered pathogen need not be synthesized de novo, but may be deliberately modified under laboratory conditions, to possess a target set of biological features.

The genetic degeneracy score can be used for determining a diagnosis of a disease in the subject. The determining the diagnosis can be based on determining an evolutionary trajectory of the sample from the subject. For example, sequencing techniques can be used to predict a future genetic state of the sample. The genetic degeneracy score can be used for determining a prognosis of the disease in the subject. For example, sequencing techniques can be used to predict a future genetic state of the sample. The determining the prognosis can be based on determining the evolutionary trajectory of the sample from the subject.

Primers can be designed based on the genetic degeneracy score, as illustrated, for instance, in FIG. 1C. At 112C of FIG. 1C, primers complementary to at least a portion of the nucleotide sequence are designed, when the genetic degeneracy is low. That is, when designing primers against a target nucleotide sequence, the primers may be designed such that they do not bind regions of the target nucleotide sequence with high degeneracy value and/or high genetic degeneracy scores, to avoid sequences that are most susceptible to mutations in the binding regions of the primers. In doing so, the designed primers are likely to bind to the target nucleotide sequence, especially if the mutations do not occur towards 3′ end of either of the primers. The designing of the primers based on the genetic degeneracy score can also be based on predicted primer binding temperatures of the primers to the nucleotide sequence. The designing of the primers based on the genetic degeneracy score can also be based on predicted secondary structure forming of the primers. That is, other features that are commonly incorporated into primer design methods can be combined with the genetic degeneracy score of the primer and/or the portion of the target nucleotide sequence the primer is designed to bind against. At 114C of FIG. 1C, the designed primers are synthesized. The primers can be sequencing primers. The primers can be amplifying primers. At 116C of FIG. 1C, the portion of the nucleotide sequence for a subject can be amplified. The amplifying primers can be used for amplifying at least a portion of the nucleotide sequence. The amplifying can comprise polymerase chain reaction. At 118C of FIG. 1C, at least the portion of the nucleotide sequence can be sequenced. The sequencing can comprise Sanger sequencing. At 120C of FIG. 1C, a disease diagnosis for the subject can be determined, based on the sequenced portion of the nucleotide sequence. The primers can be used to provide a diagnosis for the sample. The provided diagnosis can be based on the primers amplifying at least the portion of the nucleotide sequence, or based on the primers being sequencing primers, and sequencing a nucleotide sequence, e.g., an amplicon. A biomarker can be selected based on the genetic degeneracy score. For example, by analyzing (e.g., amplifying and/or sequencing) the nucleotide sequence with a low genetic degeneracy score, a biomarker can be determined or selected. Note that the nucleotide sequence can have a plurality of genetic degeneracy scores, and at least one genetic degeneracy score of the plurality of genetic degeneracy scores can be a low score.

Of note, the steps detailed in process 100A, 100B, or 100C, can, at least in part, be combined with each other. For example, the blocks of process 100B in FIG. 1B can be proceeded by at least blocks 112C to 120C of process 100C in FIG. 1C. For instance, the following sequence of steps can occur, in the following order, although not necessarily so: at 102B, nucleic acid molecules can be received; at 104B, adapters can be incorporated (e.g., ligated) onto the nucleic acid molecules; at 106B, the incorporated nucleic acid molecules can be amplified; at 108B, the amplified nucleic acid molecules can be captured; at 110B, the captured nucleic acid molecules can be sequenced to obtain sequence reads that represent the captured nucleic acid molecules; at 112B, sequence reads obtained from a sequencing method performed on a sample from a subject can be received; at 114B, the sequence reads can be aligned to a reference genome to identify alignment reads; at 116B, the alignment reads can be processed to generate a nucleotide sequence; at 118B, a window within the nucleotide can be determined; at 120B, amino acids corresponding to codons in the window can be determined; at 122B, degeneracy values for the amino acids in the window can be determined, based on the numbers of synonymous codons; at 124B, the degeneracy values in the window can be combined to determine the genetic degeneracy score; at 112C, primer complementary to at least a portion of the nucleotide sequence can be designed, when the genetic degeneracy score is low; at 114C, the designed primers can be synthesized, at 116C, at least the portion of the nucleotide sequence for a subject can be amplified; at 118C, at least the portion of the nucleotide sequence can be sequenced; at 120C, a disease diagnosis for the subject can be determined, based on the sequenced portion of the nucleotide sequence.

The methods described herein need not only apply to a received nucleotide sequence, but can also be applied to a received (e.g., input) amino acid sequence. If an amino acid sequence is received, the number of synonymous codons corresponding to each amino acid in the amino acid sequence can be determined, and that number can be used as the degeneracy value for the corresponding amino acid, or that number can be used as an input to a function that outputs the degeneracy value for the corresponding amino acid.

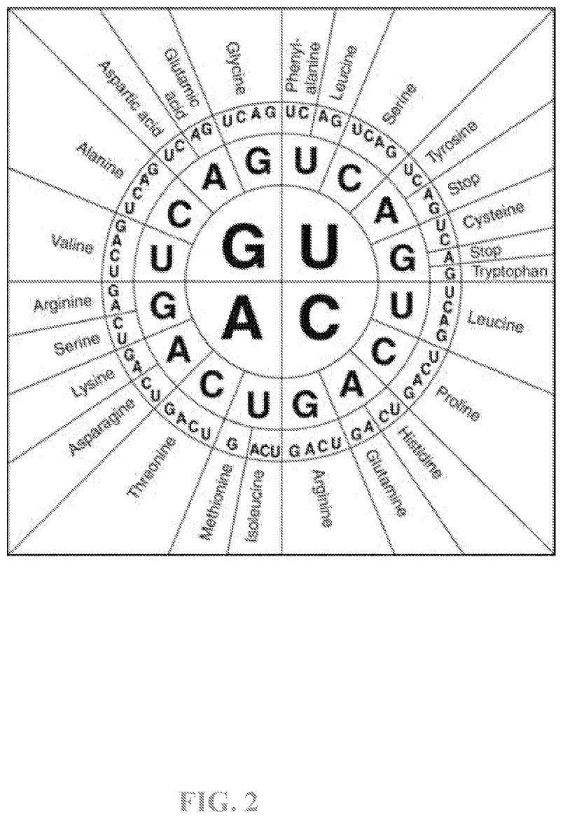

FIG. 2 depicts a non-limiting exemplary look-up table, e.g., a codon wheel, for translating a codon into an amino acid. The codon wheel can be interpreted as a look-up table that has been reformatted into a circular layout. To translate a codon into an amino acid, according to the codon wheel, the first nucleotide of the codon is selected amongst the innermost circle of four nucleotides (UCAG). Then the second nucleotide of the codon is selected from the four nucleotides (UCAG) adjacent and outwards from the first nucleotide of the codon. Then, the third nucleotide of the codon is selected from the four nucleotides (UCAG) adjacent to the second nucleotide of the codon. The amino acid closest to the selected triplet of nucleotides is the translated amino acid. In other words, the nucleotides for a given codon are read starting from the center of the circle, and then radiating outwards. Note that the nucleotide U and T are interchangeable here—U refers to the RNA sequences, which differ from DNA sequences in that the T seen in DNA sequences are replaced by U in RNA sequences. The look-up table depicted in FIG. 2 can be considered a database of nucleotide values that correspond to an amino acid value. An amino acid value can include a stop signal, in which no amino acid is produced, but instead, a stop signal that terminates the amino acid sequence is generated.

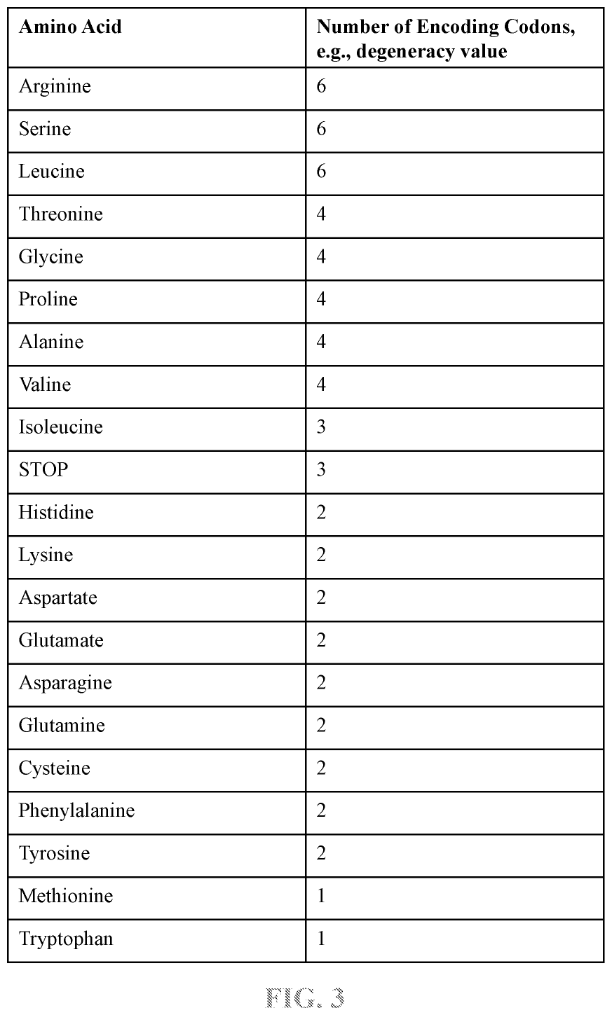

FIG. 3 depicts a non-limiting exemplary look-up table that shows the number of encoding codons for an amino acid. An amino acid value can include a stop signal (indicated in FIG. 3 as “STOP” in the amino acid column), in which no amino acid is produced, but instead, a stop signal that terminates the amino acid sequence is generated. The number of encoding codons (e.g., synonymous codons) for an amino acid can be used directly as the degeneracy value for a given amino acid. Alternatively, the number of encoding codons (e.g., synonymous codons) for an amino acid can be inputted into a transformation function, to yield a degeneracy value. The degeneracy value can then be combined into a genetic degeneracy score for a window applied to the nucleotide sequence. The look-up table in FIG. 3 can be considered a database of values, e.g., numbers of encoding codons for an amino acid, associated with amino acids.

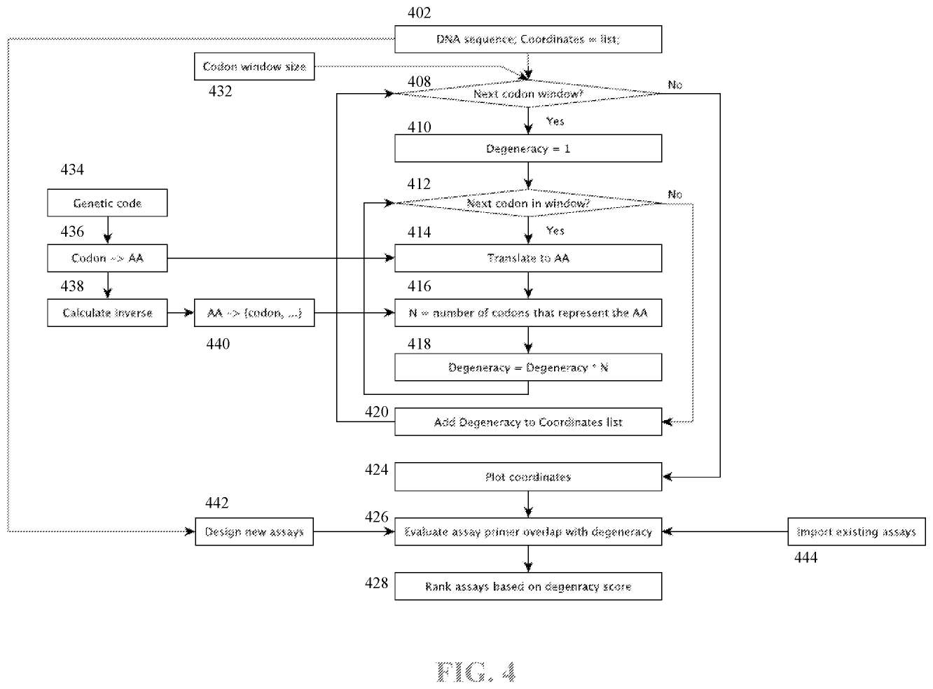

FIG. 4 depicts a non-limiting exemplary flowchart for determining and using the genetic degeneracy score for a nucleotide sequence. Box 402 specifies preparing a set of nucleic acid sequence coordinates in a list data structure, for the first frame, i.e., window. The output of box 402 is then passed to box 408, which provides a decision point, and asks if there are any remaining codons in the window. If no, the output of box 408 is passed to box 424, and the coordinates are plotted, the output of which is passed to box 426, which evaluates the assay primer overlap with degeneracy. Box 426 also integrates outputs from box 442, which specifies designing new assays, and box 444, which imports existing assays. The output of box 426 is passed to box 428, which ranks the assays based on the genetic degeneracy score. If the decision point at box 408 results in a yes, the output is passed to box 410, which from the DNA sequence, gets a frame sequence, starting at position 1. The codon window size as specified in box 432 is then incorporated into box 408, which provides a decision point, and asks if the next codon in the window can be analyzed. If no, then the output is passed to box 424, and the frame stops sliding across the nucleotide sequence and the coordinates are plotted. The output of box 410 is passed to box 412, which provides a decision point, and asks if the next codon in the window can be analyzed. If no, the frame number, the nucleotide position, and the degeneracy values are added to the coordinates in box 420, and the next codon window is assessed, as detailed in box 408. If the output of box 412 is affirmative, then the codon in the window is translated into an amino acid at box 414, and the translation is the result of using the genetic code as specified in box 434, to convert a codon into an amino acid, as specified in box 436, and the amino acid is provided to box 414 and incorporated. Once the sequence is translated to an amino acid at box 414, at box 416, the number of codons that represent the translated amino acid is assigned to a variable, N. The number of codons that represent the translated amino acid at box 416 is based on box 438, which determines a codon, from a given amino acid, and then passes the determined codon to box 440, where the number of codons that can represent the amino acid is determined, and that number of codons is passed to box 416, and the number of codons is the value assigned to the variable, N. Then, at box 418, the degeneracy value is computed by multiplying the value of N by the value of the product variable. Once the degeneracy value is determined, the next codon is assessed, as seen in box 412, and again, the degeneracy value is determined. Once the degeneracy values are determined for all the codons in the window, the degeneracy values are combined, e.g., multiplied together, into genetic degeneracy score, and that score is plotted. Then the window slides to the next position, until the window has slid across the entire nucleotide sequence. The genetic degeneracy scores for the nucleotide sequence are plotted at box 424, and primer overlap with the degeneracy scores are assessed, for primers from existing assays, as seen in box 444, and for primers from new assays, as seen in box 442. The assays can then be ranked based on the degeneracy score, in box 428.

### Systems

Also disclosed herein are systems designed to implement any of the disclosed methods for determining a genetic degeneracy score from a sample from a subject. The systems may comprise, e.g., one or more processors, and a memory unit communicatively coupled to the one or more processors and configured to store instructions that, when executed by the one or more processors, cause the system to: receive a nucleotide sequence; determine a window within the nucleotide sequence; determine one or more amino acids corresponding to one or more codons in the window; determine one or more degeneracy values for the one or more amino acids in the window; and combine the one or more degeneracy values in the window to determine the genetic degeneracy score. The system may comprise further instructions that, when executed by the one or more processors, cause the system to slide the window by at least one nucleotide across the nucleotide sequence, to generate a plurality of genetic degeneracy scores. The system may comprise even further instructions that, when executed by the one or more processors, cause the system to combine the plurality of genetic degeneracy scores into a final genetic degeneracy score.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

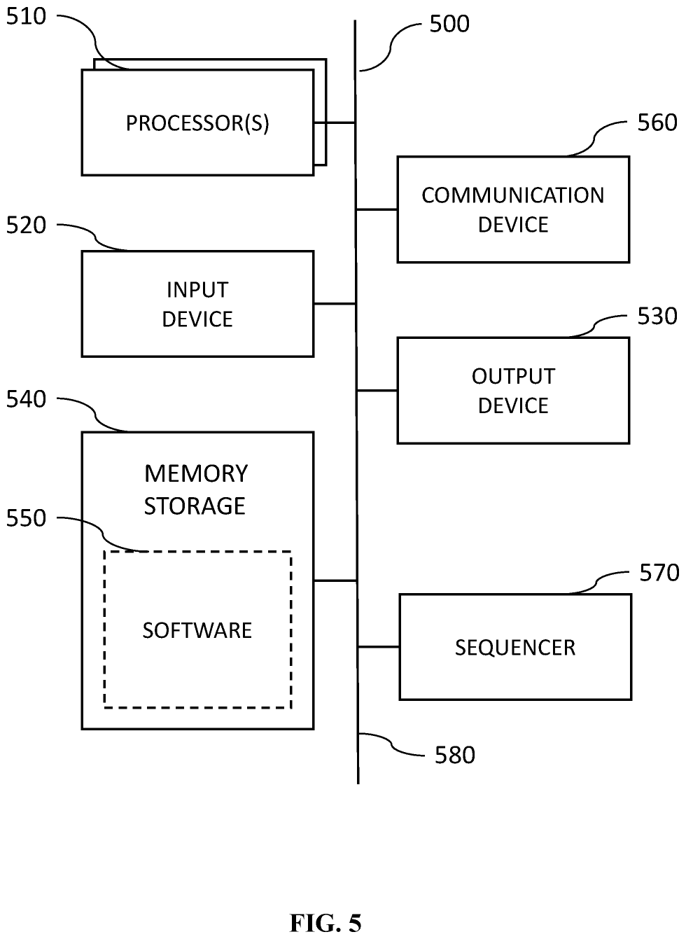

FIG. 5 illustrates an example of a computing device or system in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more processor(s) 510, input devices 520, output devices 530, memory or storage devices 540, communication devices 560, and nucleic acid sequencers 570. Software 550 residing in memory or storage device 540 may comprise, e.g., an operating system as well as software for executing the methods described herein. Input device 520 and output device 530 can generally correspond to those described herein, and can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage (e.g., an electrical, magnetic or optical memory including a RAM (volatile and non-volatile), cache, hard drive, or removable storage disk). Communication device 560 can include any suitable device capable of transmitting and receiving signals over a network, such as a network interface chip or device. The components of the computer can be connected in any suitable manner, such as via a wired media (e.g., a physical system bus 580, Ethernet connection, or any other wire transfer technology) or wirelessly (e.g., Bluetooth®, Wi-Fi®, or any other wireless technology).

Software module 550, which can be stored as executable instructions in storage 540 and executed by processor(s) 510, can include, for example, an operating system and/or the processes that embody the functionality of the methods of the present disclosure (e.g., as embodied in the devices as described herein).

Software module 550 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 540, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

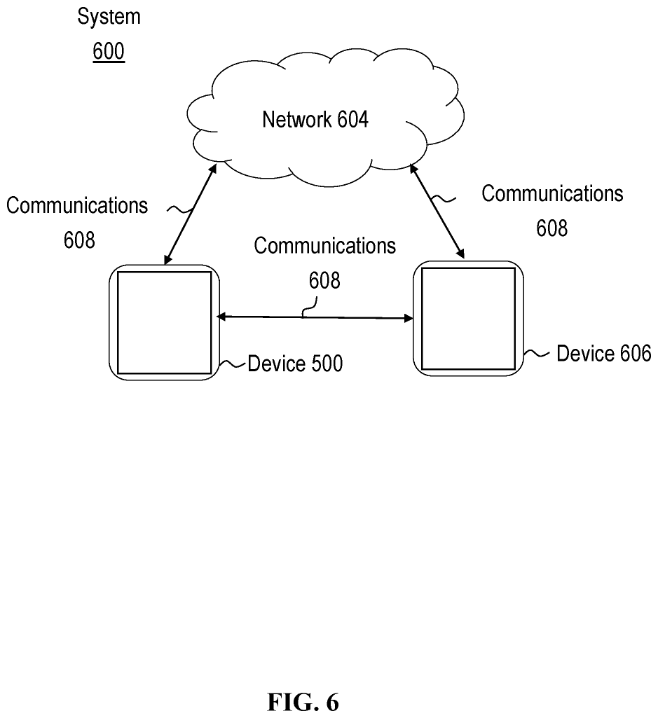

Device 500 may be connected to a network (e.g., network 604, as shown in FIG. 6 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can be implemented using any operating system, e.g., an operating system suitable for operating on the network. Software module 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 510.

Device 500 can further include a sequencer 570, which can be any suitable nucleic acid sequencing instrument.

FIG. 6 illustrates an example of a computing system in accordance with one embodiment. In system 600, device 500 (e.g., as described above and illustrated in FIG. 5) is connected to network 604, which is also connected to device 606. In some embodiments, device 606 is a nucleic acid sequencer.

Devices 500 and 606 may communicate, e.g., using suitable communication interfaces via network 604, such as a Local Area Network (LAN), Virtual Private Network (VPN), or the Internet. In some embodiments, network 604 can be, for example, the Internet, an intranet, a virtual private network, a cloud network, a wired network, or a wireless network. Devices 500 and 606 may communicate, in part or in whole, via wireless or hardwired communications, such as Ethernet, IEEE 802.11b wireless, or the like. Additionally, devices 500 and 606 may communicate, e.g., using suitable communication interfaces, via a second network, such as a mobile/cellular network. Communication between devices 500 and 606 may further include or communicate with various servers such as a mail server, mobile server, media server, telephone server, and the like. In some embodiments, Devices 500 and 606 can communicate directly (instead of, or in addition to, communicating via network 604), e.g., via wireless or hardwired communications, such as Ethernet, IEEE 802.11b wireless, or the like. In some embodiments, devices 500 and 606 communicate via communications 608, which can be a direct connection or can occur via a network (e.g., network 604).

One or all of devices 500 and 606 generally include logic (e.g., http web server logic) or are programmed to format data, accessed from local or remote databases or other sources of data and content, for providing and/or receiving information via network 604 according to various examples described herein.

### EXAMPLES

### Example 1

This section provides an example of determining genetic degeneracy scores, based on the first 12 nucleotides of the fiber gene of Human adenovirus B55 (NCBI accession no. FJ643676.1 region from position 30775 to 31752).

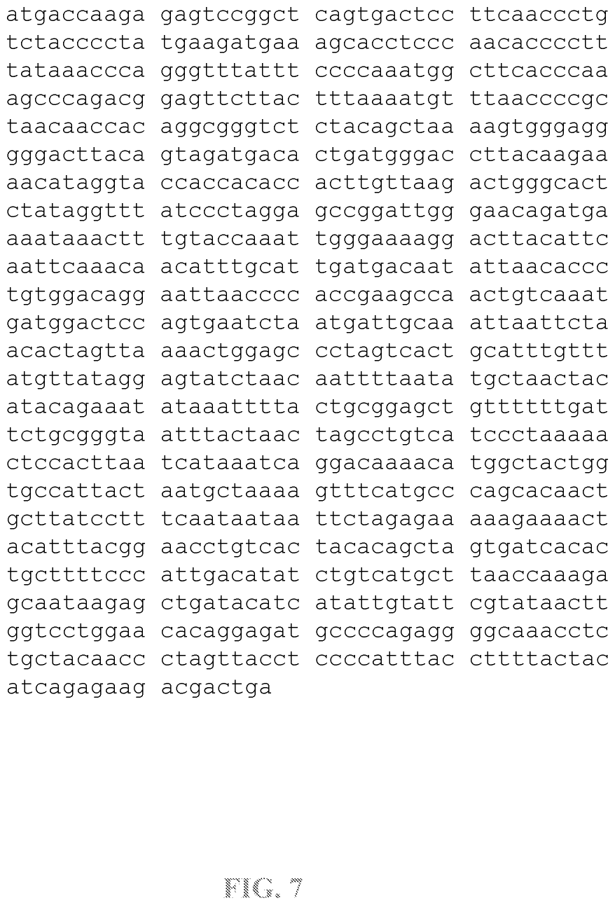

First, a nucleotide sequence was received. The received nucleotide sequence was the first 12 nucleotides of the fiber gene of Human adenvirus B55, for which the entire sequence is depicted in FIG. 7. More specifically, FIG. 7 depicts the (+) strand, i.e., sense strand, of a double-stranded DNA sequence, from 5′ to 3′, as is convention.

The determining, e.g., computing, of the genetic degeneracy score for the received nucleotide sequence can be defined more precisely, in relation to mathematical expressions—and more specifically, with regards to set-builder and sequence-builder notations. Further, as expressed herein, codons are represented interchangeably as text or character sequences (e.g, CAT and (C, A, T) are equivalent). In the case of the present example, the canonical DNA and amino acid alphabets (where ‘*’ indicates the stop signal) were first defined respectively as [symbol] and [symbol] :

$$= \{ A , C , G , T \}$$

$$= \{ A , C , D , E , F , G , H , I , K , L , M , N , P , Q , R , S , T , V , W , Y , * \}$$

In this example, the function g maps the amino acid k to the set of codons that encode it. The optional parameter d is the set corresponding to unknown (amino acid) symbols, which is the empty set Ø by default. Thus, the inverse of the given genetic code that maps each amino acid (and stop signal optionally) to its corresponding set of codons was calculated:

$$g ( k , d = ∅ ) = \{ \begin{matrix}\{ ( G , C , A ) , ( G , C , C ) , ( G , C , G ) , ( G , C , T ) \} , & k = A \\ \{ ( T , G , C ) , ( T , G , T ) \} , & k = C \\ \{ ( G , A , C ) , ( G , A , T ) \} , & k = D \\ \{ ( G , A , A ) , ( G , A , G ) \} , & k = E \\ \{ ( T , T , C ) , ( T , T , T ) \} , & k = F \\ \{ ( G , G , A ) , ( G , G , C ) , ( G , G , G ) , ( G , G , T ) \} , & k = G \\ \{ ( C , A , C ) , ( C , A , T ) \} , & k = H \\ \{ ( A , T , A ) , ( A , T , C ) , ( A , T , T ) \} , & k = I \\ \{ ( A , A , A ) , ( A , A , G ) \} , & k = K \\ \{ ( C , T , A ) , ( C , T , C ) , ( C , T , G ) , ( C , T , T ) , ( T , T , A ) , ( T , T , G ) \} , & k = L \\ \{ ( A , T , G ) \} , & k = M \\ \{ ( A , A , C ) , ( A , A , T ) \} , & k = N \\ \{ ( C , C , A ) , ( C , C , C ) , ( C , C , G ) , ( C , C , T ) \} , & k = P \\ \{ ( C , A , A ) , ( C , A , G ) \} , & k = Q \\ \{ ( A , G , A ) , ( A , G , G ) , ( C , G , A ) , ( C , G , C ) , ( C , G , G ) , ( C , G , T ) \} , & k = R \\ \{ ( A , G , C ) , ( A , G , T ) , ( T , C , A ) , ( T , C , C ) , ( T , C , G ) , ( T , C , T ) \} , & k = S \\ \{ ( A , C , A ) , ( A , C , C ) , ( A , C , G ) , ( A , C , T ) \} , & k = T \\ \{ ( G , T , A ) , ( G , T , C ) , ( G , T , G ) , ( G , T , T ) \} , & k = V \\ \{ ( T , G , G ) \} , & k = W \\ \{ ( T , A , C ) , ( T , A , T ) \} , & k = Y \\ \{ ( T , A , A ) , ( T , G , A ) , ( T , A , G ) \} , & k = * \\ d , & otherwise\end{matrix}$$

The function that computes the degeneracy for a given codon was then calculated, which returned the total number of codons that mapped to the same amino acid or a default value, such as d=1, if the codon was unknown. Accordingly, function h calculates degeneracy for the given codon k and default degeneracy value d. The function h builds the set of all codons that map to the amino acid defined by k and submits the cardinality of that set to the function ƒ, which returns the value (x) unchanged if it is greater than 0 or d otherwise.

$$f ( x , d ) = \{ \begin{matrix}d , & x \leq 0 \\ x , & x > 0\end{matrix}$$

$$h ( k , d = 1 ) = f ( \vert \{ e \vert a ∈ ∧ e ∈ g ( a ) ∧ k ∈ g ( a ) \} \vert , d )$$

A DNA sequence of length n was then defined:

$$n ∈ , s ∈$$

$$n = 12 , s = ( A , T , G , A , C , C , A , A , G , A , G , A )$$

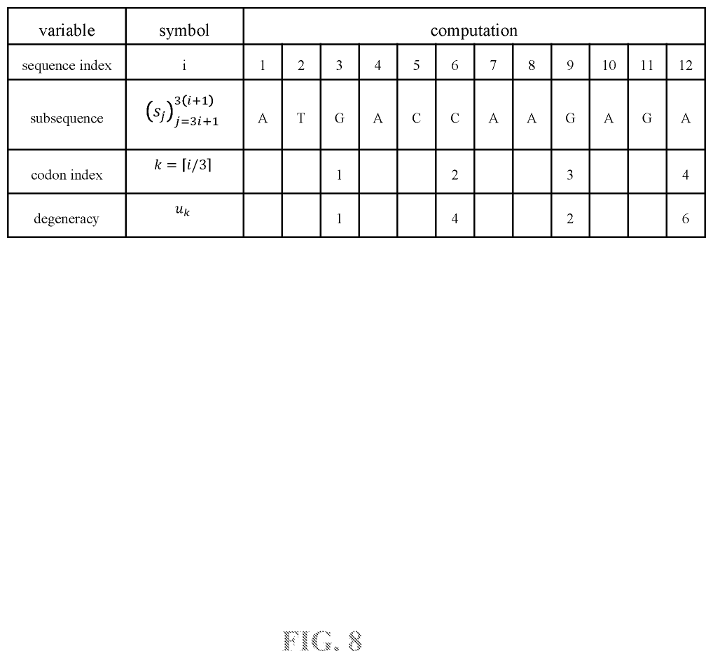

The sequence of codon degeneracy values at each codon index in the sequence was then built, the results of which can also be seen in FIG. 8:

$$u = ( h ( {( {s}_{j} )}_{j = 1 + i - 3}^{i} ) \vert 3 \leq i \leq \vert s \vert ∧ 3 \vert i )$$

$$u = ( h ( ( A , T , G ) ) , h ( ( A , C , C ) ) , h ( ( A , A , G ) ) , h ( ( A , G , A ) ) = ( 1 , 4 , 2 , 6 )$$

The window length (i.e., window size) was then defined as an odd number to compute the sequence of local, sliding-window degeneracy scores:

$$w ∈ \{ 2 i + 1 \vert i ∈ \}$$

$$w = 3$$

The window length of w=3 can be interpreted as a window size of 3, which for the given natural, standard genetic code is a window length of 3 codons or 3 amino acids or 9 nucleotides.

The number of adjacent degeneracy values to build the window at each sequence index of u was then calculated:

$$m = ⌊ \frac{w}{2} ⌋ = ⌊ \frac{3}{2} ⌋ = 1$$

The window was then slid across the sequence of computed degeneracy values u, based on the adjacent number of values m. The degeneracy score at each sliding window position was calculated as the sequence d:

$$d = ( {\prod }_{k = max ( 1 , i - m )}^{min ( i + m , \vert u \vert )} {u}_{k} \vert 1 \leq i \leq \vert u \vert )$$

$$d = ( ( {u}_{1} {u}_{2} ) , ( {u}_{1} {u}_{2} {u}_{3} ) , ( {u}_{2} {u}_{3} {u}_{4} ) , ( {u}_{3} {u}_{4} ) )$$

$$d = ( ( 1 \times 4 ) , ( 1 \times 4 \times 2 ) , ( 4 \times 2 \times 6 ) , ( 2 \times 6 ) ) = ( 4 , 8 , 48 , 12 )$$

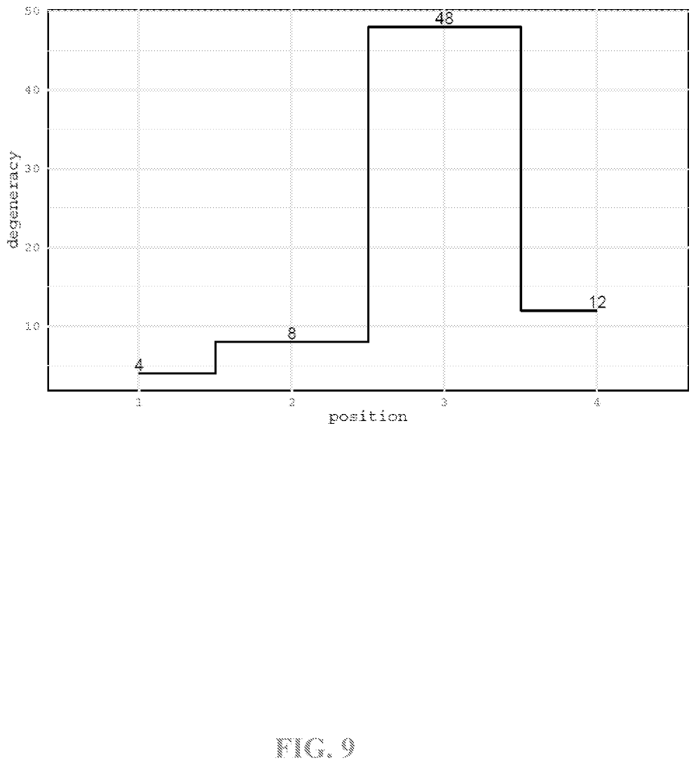

The values of the genetic degeneracy scores, d, for the received nucleotide sequence are plotted in FIG. 9.

The global degeneracy score d_{G} was also computed, which is equal to the total number of alternate DNA sequences that encode the same protein sequence. This is equivalent to setting the sliding-window length w=1. It follows that when w=1, then d=u. For the received nucleotide sequence, for which u=(1, 4, 2, 6), d_{G} was then calculated as follows:

$$w = 1 , m = ⌊ \frac{w}{2} ⌋ = ⌊ \frac{1}{2} ⌋ = 0$$

$${d}_{G} = \prod ( {\prod }_{k = max ( 1 , i - m )}^{min ( i + m , \vert u \vert )} {u}_{k} \vert 1 \leq i \leq \vert u \vert ) = \prod ( 1 , 4 , 2 , 6 ) = ( 1 \times 4 \times 2 \times 6 ) = 48$$

### Example 2

The present Example remains concerned with the DNA sequence of the Human adenovirus B55 strain, as seen in the previous Example. Unlike the previous Example, the received nucleotide sequence for the present Example concerns the entire fiber gene of the Human adenovirus B55 strain.

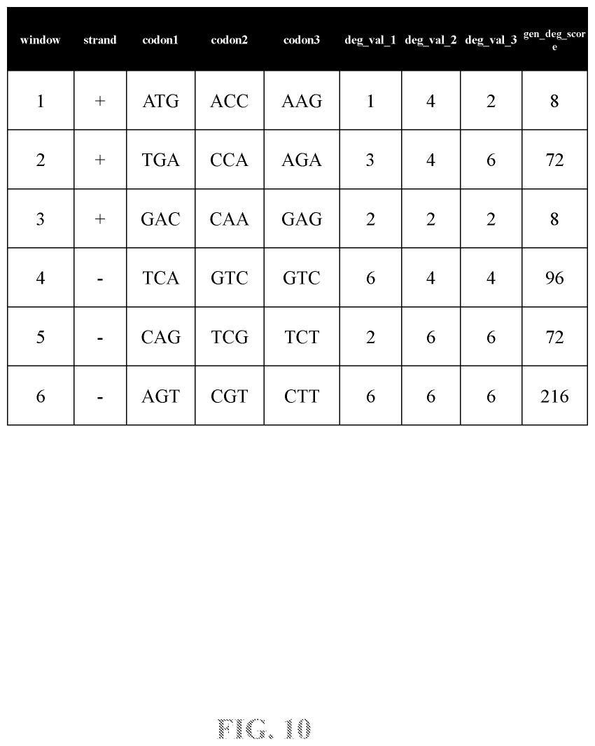

FIG. 10 shows data related to the determining of the degeneracy values and the genetic degeneracy scores for the received nucleotide sequence. Genetic degeneracy scores are determined for the nucleotide sequence, given different starting positions. The sense strand is examined for three different starting window positions, and the antisense strand is examined for three different starting window positions.

In the first window position, on the (+) strand, i.e., the sense strand, the codons captured within the window are depicted, i.e., the sequences for codon 1, codon 2, and codon 3. The sequence for codon 1 was ATG, which translated to a corresponding amino acid of methionine, which could be encoded by only 1 codon sequence. The sequence for codon 2, which was directly 3′ of codon 1, was ACC, which translated to a corresponding amino acid of threonine, which could be encoded by 4 different codon sequences. The sequence for codon 3, which was directly 3′ of codon 2, was AAG, which translated to a corresponding amino acid of lysine, which could be encoded by 2 different codon sequences. The look-up table, e.g., codon wheel, shown in FIG. 2 could be referenced to determine the correspondences between the nucleotide codons and their corresponding amino acids, for the naturally occurring genetic code. The table shown in FIG. 3 could be referenced to determine the number of synonymous codons possible for a given amino acid, which could be incorporated into determining the degeneracy values and/or the genetic degeneracy score. For the example described herein, the number of synonymous codons for an encoded amino acid within the window is identical to the degeneracy value for that codon or amino acid, but other implementations of the methods described herein need not be limited as such. For example, alternative implementations may scale, e.g., normalize, the degeneracy value by a common scalar factor, or in general, transform the degeneracy value via a transformation function. In this example, the degeneracy value for window 1 was 1, for window 2 was 6, and for window 3 was 2. These degeneracy values for window 1 were then combined—in this case, multiplied together—to determine a genetic degeneracy score of 8 for window 1 (i.e., 1*4*2=8).

In the next iteration of the present example for the method, the window was slid over one nucleotide towards 3′ end of the nucleotide sequence. The same steps were performed, for which the look-up tables shown in FIGS. 2 and 3 can be referenced to determine the degeneracy values and genetic degeneracy score of the new window, window 2. Again, 9 nucleotides, e.g., 3 codons, were captured within the window. In window 2: codon 1 was TGA, which translated to a stop codon, which could be encoded by 3 codons, and a degeneracy value of 3 was assigned; codon 2 was CCA, which translated to proline, which could be encoded by 4 codons, and a degeneracy value of 4 was assigned; codon 3 was AGA, which translated to arginine, which could be encoded by 6 codons, and a degeneracy value of 6 was assigned. The degeneracy scores for window 2 were multiplied together, to determine a genetic degeneracy score of 16 for window 2 (i.e., 3*4*6=72).

In the next iteration of the present example for the method, the window was slid over one nucleotide towards 3′ end of the nucleotide sequence. The same steps were performed, for which the look-up tables shown in FIGS. 2 and 3 can be referenced to determine the degeneracy values and genetic degeneracy score of the new window, window 3. Again, 9 nucleotides, e.g., 3 codons, were captured within the window. In window 3: codon 1 was GAC, which translated to aspartate, which could be encoded by 2 codons, and a degeneracy value of 2 was assigned; codon 2 was CAA, which translated to a glutamine codon, which could be encoded by 2 codons, and a degeneracy value of 2 was assigned; codon 3 was GAAG, which translated to glutamate, which could be encoded by 2 codons, and a degeneracy value of 2 was assigned. The degeneracy scores for window 3 were multiplied together, to determine a genetic degeneracy score of 8 for window 3 (i.e., 2*2*2=8).

Windows 4, 5, and 6 relate to the (−) strand (i.e., the anti-sense strand) of the nucleic acid sequence. The anti-sense strand is the reverse complement of the sense strand, for which the sequence is depicted in FIG. 7. As is convention, the sequence of the anti-sense strand is depicted from 5′ to 3′ in FIG. 10.

In the next iteration of the present example for the method, the window was slid over one nucleotide towards 3′ end of the nucleotide sequence. The same steps were performed, for which the look-up tables shown in FIGS. 2 and 3 can be referenced to determine the degeneracy values and genetic degeneracy score of the new window, window 4. Again, 9 nucleotides, e.g., 3 codons, were captured within the window. In window 4: codon 1 was TCA, which translated to serine, which could be encoded by 6 codons, and a degeneracy value of 6 was assigned; codon 2 was GTC, which translated to valine, which could be encoded by 4 codons, and a degeneracy value of 4 was assigned; codon 3 was GTC, which translated to valine, which could be encoded by 4 codons, and a degeneracy value of 4 was assigned. The degeneracy scores for window 4 were multiplied together, to determine a genetic degeneracy score of 96 for window 4 (i.e., 6*4*4=96).

In the next iteration of the present example for the method, the window was slid over one nucleotide towards 3′ end of the nucleotide sequence. The same steps were performed, for which the look-up tables shown in FIGS. 2 and 3 can be referenced to determine the degeneracy values and genetic degeneracy score of the new window, window 5. Again, 9 nucleotides, e.g., 3 codons, were captured within the window. In window 5: codon 1 was CAG, which translated to glutamine, which could be encoded by 2 codons, and a degeneracy value of 2 was assigned; codon 2 was TCG, which translated to serine, which could be encoded by 6 codons, and a degeneracy value of 6 was assigned; codon 3 was TCT, which translated to serine, which could be encoded by 6 codons, and a degeneracy value of 6 was assigned. The degeneracy scores for window 5 were multiplied together, to determine a genetic degeneracy score of 24 for window 5 (i.e., 2*6*6=72).

In the next iteration of the present example for the method, the window was slid over one nucleotide towards 3′ end of the nucleotide sequence. The same steps were performed, for which the look-up tables shown in FIGS. 2 and 3 can be referenced to determine the degeneracy values and genetic degeneracy score of the new window, window 6. Again, 9 nucleotides, e.g., 3 codons, were captured within the window. In window 6: codon 1 was AGT, which translated to isoleucine, which could be encoded by 6 codons, and a degeneracy value of 6 was assigned; codon 2 was CGT, which translated to arginine, which could be encoded by 6 codons, and a degeneracy value of 6 was assigned; codon 3 was CTT, which translated to leucine, which could be encoded by 6 codons, and a degeneracy value of 6 was assigned. The degeneracy scores for window 6 were multiplied together, to determine a genetic degeneracy score of 18 for window 6 (i.e., 6*6*6=216).

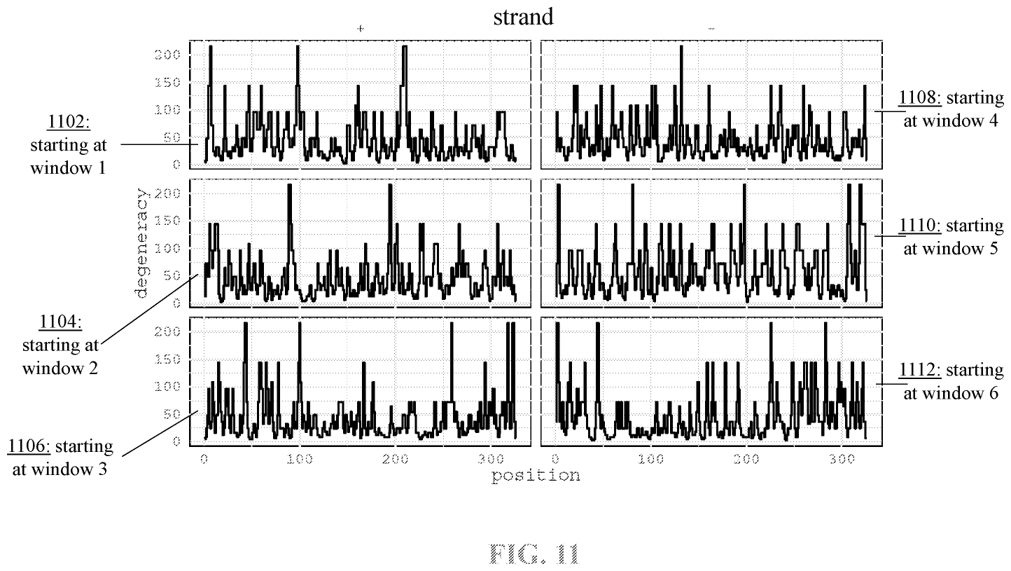

The determined genetic degeneracy scores depicted in FIG. 11, were based on the nucleotide sequence depicted in FIG. 7, and were graphed across 6 plots in FIG. 11. The plots in FIG. 11 were generated from determining genetic degeneracy scores upon sliding the window across the nucleotide sequence in FIG. 7, where each plots begins the window sliding at a different initial position. Plots 1102, 1104, and 1106 correspond to genetic degeneracy scores determined for the sense strand of the nucleotide sequence depicted in FIG. 7. Plots 1108, 1110, and 1112 correspond to genetic degeneracy scores determined for the antisense strand of the nucleotide sequence depicted in FIG. 7. Plot 1102 depicts genetic degeneracy scores for which the first plotted genetic degeneracy score corresponds to the genetic degeneracy score determined in window 1 of FIG. 10, i.e., 8. The remaining genetic degeneracy scores plotted on plot 1102 were determined upon sliding the window one nucleotide to the right, for each genetic degeneracy score. Plot 1104 depicts genetic degeneracy scores for which the first plotted genetic degeneracy score corresponds to the genetic degeneracy score determined in window 2 of FIG. 10, i.e., 72. The remaining genetic degeneracy scores plotted on plot 1104 were determined upon sliding the window one nucleotide to the right, for each genetic degeneracy score. Plot 1106 depicts genetic degeneracy scores for which the first plotted genetic degeneracy score corresponds to the genetic degeneracy score determined in window 3 of FIG. 10, i.e., 8. The remaining genetic degeneracy scores plotted on plot 1106 were determined upon sliding the window one nucleotide to the right, for each genetic degeneracy score. Plot 1108 depicts genetic degeneracy scores for which the first plotted genetic degeneracy score corresponds to the genetic degeneracy score determined in window 4 of FIG. 10, i.e., 96. The remaining genetic degeneracy scores plotted on plot 1108 were determined upon sliding the window one nucleotide to the right, for each genetic degeneracy score. Plot 1110 depicts genetic degeneracy scores for which the first plotted genetic degeneracy score corresponds to the genetic degeneracy score determined in window 5 of FIG. 10, i.e., 72. The remaining genetic degeneracy scores plotted on plot 1110 were determined upon sliding the window one nucleotide to the right, for each genetic degeneracy score. Plot 1112 depicts genetic degeneracy scores for which the first plotted genetic degeneracy score corresponds to the genetic degeneracy score determined in window 6 of FIG. 10, i.e., 216. The remaining genetic degeneracy scores plotted on plot 1112 were determined upon sliding the window one nucleotide to the right, for each genetic degeneracy score.

### Exemplary Embodiments

Exemplary embodiments of the methods and systems described herein include:

Embodiment 1. A method of determining a genetic degeneracy score, comprising:
receiving a nucleotide sequence, by one or more processors;
determining a window within the nucleotide sequence, by the one or more processors;
determining one or more amino acids corresponding to one or more codons in the window, by the one or more processors;
determining, by the one or more processors, one or more degeneracy values for the one or more amino acids in the window; and
combining the one or more degeneracy values in the window to determine the genetic degeneracy score, by the one or more processors.

Embodiment 2. The method of embodiment 1, further comprising sliding the window by at least one nucleotide across the nucleotide sequence, to generate a plurality of genetic degeneracy scores.

Embodiment 3. The method of embodiment 2, wherein further comprising combining the plurality of genetic degeneracy scores into a final genetic degeneracy score.

Embodiment 4. The method of any of embodiments 1-3, wherein the genetic degeneracy score is used for identifying a conserved genetic sequence from a sample from a subject.

Embodiment 5. The method of any of embodiments 1-4, wherein the identified conserved genetic sequence is used for detecting a pathogen.

Embodiment 6. The method of embodiment 5, wherein the pathogen is an engineered pathogen.

Embodiment 7. The method of any of embodiments 1-6, wherein the genetic degeneracy score is used for determining a diagnosis of a disease in the subject.

Embodiment 8. The method of embodiment 7, wherein the determining the diagnosis is based on determining an evolutionary trajectory of the sample from the subject.

Embodiment 9. The method of any of embodiments 1-8, wherein the genetic degeneracy score is used for determining a prognosis of the disease in the subject.

Embodiment 10. The method of embodiment 9, wherein the determining the prognosis is based on determining the evolutionary trajectory of the sample from the subject.

Embodiment 11. The method of any of embodiments 4-10, wherein the sample is a cancer sample.

Embodiment 12. The method of any of embodiments 1-11, wherein primers are designed based on the genetic degeneracy score.

Embodiment 13. The method of embodiment 12, wherein the designing the primers based on the genetic degeneracy score is also based on predicted primer binding temperatures of the primers to the nucleotide sequence.

Embodiment 14. The method of embodiment 12 or 13, wherein the designing the primers based on the genetic degeneracy score is also based on predicted secondary structure forming of the primers.

Embodiment 15. The method of any of embodiments 12-14, wherein the primers are sequencing primers.

Embodiment 16. The method of any of embodiments 12-15, wherein the primers are amplifying primers.

Embodiment 17. The method of embodiment 16, wherein the amplifying primers are used for amplifying at least a portion of the nucleotide sequence.

Embodiment 18. The method of embodiment 17, wherein the amplifying comprises polymerase chain reaction.

Embodiment 19. The method of any of embodiments 12-18, wherein the primers are used to provide a diagnosis for the sample.

Embodiment 20. The method of any of embodiments 1-19, wherein a biomarker is selected based on the genetic degeneracy score.

Embodiment 21. The method of any of embodiments 1-20, wherein the nucleotide sequence is an engineered nucleotide sequence.

Embodiment 22. The method of any of embodiments 1-21, wherein the nucleotide sequence is a predicted nucleotide sequence.

Embodiment 23. The method of any of embodiments 4-22, wherein the nucleotide sequence is based on the sample from the subject.

Embodiment 24. The method of any of embodiments 4-23, wherein the sample comprises a liquid biopsy sample.

Embodiment 25. The method of any of embodiments 4-24, wherein the liquid biopsy sample comprises blood, plasma, cerebrospinal fluid, sputum, stool, urine, sweat, or saliva.

Embodiment 26. The method of any of embodiments 4-25, wherein the sample comprises a tissue biopsy sample.

Embodiment 27. The method of any of embodiments 4-26, wherein a genetic code for the subject is an artificial genetic code.

Embodiment 28. The method of any of embodiments 1-27, wherein the method is iterative with respect to the determining the window, the determining the one or more amino acids, the determining the one or more degeneracy values, or the combining the one or more degeneracy values.

Embodiment 29. The method of any of embodiments 2-28, wherein the method is iterative with respect to the sliding the window.

Embodiment 30. The method of embodiment 28 or 29, wherein the iterative method stops after a predetermined number of iterations.

Embodiment 31. The method of any of embodiments 28-30, wherein the iterative method stops after the sliding the window comprises sliding across the nucleotide sequence in its entirety.

Embodiment 32. The method of any of embodiments 1-31, wherein a first length of the window for a first iteration of the method can overlap with a second length of the window for a second iteration of the method.

Embodiment 33. The method of any of embodiments 1-32, further comprising using a codon look-up table for the determining the one or more amino acids corresponding to the one or more codons in the window.

Embodiment 34. The method of any of embodiments 1-33, further comprising using the codon look-up table for the determining the one or more degeneracy values.

Embodiment 35. The method of any of embodiments 1-34, wherein end regions of the nucleotide sequence are padded with padding values.

Embodiment 36. The method of embodiment 35, wherein the end regions are shorter in length than a length of the window.

Embodiment 37. The method of embodiment 35 or 36, wherein the padding values comprise indeterminate values.

Embodiment 38. The method of any of embodiments 35-37, wherein the length of the window in nucleotides is divisible by a length of a codon from the one or more codons, in nucleotides.

Embodiment 39. The method of any of embodiments 35-38, wherein the length of the window in nucleotides is divisible by three.

Embodiment 40. The method of any of embodiments 35-39, wherein the length of the window in nucleotides is at most equal to a length of the nucleotide sequence.

Embodiment 41. The method of any of embodiments 1-40, wherein a number of determined degeneracy values is fewer than the length of the window in nucleotides divided by the length of the codon.

Embodiment 42. The method of any of embodiments 1-41, wherein one degeneracy value from the one or more degeneracy values is determined for each codon in the one or more codons.

Embodiment 43. The method of any of embodiments 1-42, wherein the combining comprises multiplying together the one or more degeneracy values in the window.

Embodiment 44. The method of any of embodiments 1-43, wherein the combining comprises adding together the one or more degeneracy values in the window.

Embodiment 45. The method of any of embodiments 1-44, wherein a degeneracy value from the one or more degeneracy values ranges between 1 and 216.

Embodiment 46. The method of any of embodiments 1-45, wherein the degeneracy value is an irrational number, a rational number, an integer, a whole number, or a natural number.

Embodiment 47. The method of any of embodiments 1-46, wherein the degeneracy value is a computed integer or a computed float.

Embodiment 48. The method of any of embodiments 1-47, wherein the genetic degeneracy score is combined with a determining a molecular clock rate for the nucleotide sequence.

Embodiment 49. A method of determining a genetic degeneracy score for a sample from a subject, comprising:
receiving nucleic acid molecules obtained from the sample from the subject;
incorporating one or more adapters onto one or more nucleic acid molecules from the nucleic acid molecules;
amplifying the one or more incorporated nucleic acid molecules from the nucleic acid molecules;
capturing the amplified nucleic acid molecules from the incorporated nucleic acid molecules;
sequencing, by a sequencer, the captured nucleic acid molecules to obtain sequence reads that represent the captured nucleic acid molecules;
receiving sequence reads obtained from a sequencing method performed on the sample from the subject, by one or more processors;
aligning the sequence reads to a reference genome to identify alignment reads, by the one or more processors;
processing the alignment reads to generate a nucleotide sequence, by the one or more processors;
determining a window within the nucleotide sequence, by the one or more processors;
determining one or more amino acids corresponding to one or more codons in the window, by the one or more processors;
determining, by the one or more processors, one or more degeneracy values for the one or more amino acids in the window; and
combining the one or more degeneracy values in the window to determine the genetic degeneracy score, by the one or more processors

Embodiment 50. The method of embodiment 49, further comprising sliding the window by at least one nucleotide across the nucleotide sequence.

Embodiment 51. The method of any of embodiments 4-50, wherein the subject is a human.

Embodiment 52. A system comprising:
one or more processors; and
a memory communicatively coupled to the one or more processors and configured to store instructions that, when executed by the one or more processors, cause the system to:
   receive a nucleotide sequence;
   determine a window within the nucleotide sequence;
   determine one or more amino acids corresponding to one or more codons in the window;
   determine one or more degeneracy values for the one or more amino acids in the window; and
   combine the one or more degeneracy values in the window to determine the genetic degeneracy score.

Embodiment 53. The system of embodiment 52, comprising further instructions that, when executed by the one or more processors, cause the system to slide the window by at least one nucleotide across the nucleotide sequence, to generate a plurality of genetic degeneracy scores.

Embodiment 54. The system of embodiment 53, comprising further instructions that, when executed by the one or more processors, cause the system to combine the plurality of genetic degeneracy scores into a final genetic degeneracy score.

Embodiment 55. A non-transitory computer-readable storage medium storing one or more programs, the one or more programs comprising instructions, which when executed by one or more processors of a system, cause the system to:
receive a nucleotide sequence;
determine a window within the nucleotide sequence;
determine one or more amino acids corresponding to one or more codons in the window;
determine one or more degeneracy values for the one or more amino acids in the window; and
combine the one or more degeneracy values in the window to determine the genetic degeneracy score.

Embodiment 56. The non-transitory computer-readable storage medium of embodiment 55, further comprising instructions that, when executed by the one or more processors, cause the system to slide the window by at least one nucleotide across the nucleotide sequence, to generate a plurality of genetic degeneracy scores.

Embodiment 57. The non-transitory computer-readable storage medium of embodiment 56, further comprising instructions that, when executed by the one or more processors, cause the system to combine the plurality of genetic degeneracy scores into a final genetic degeneracy score.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method of determining a genetic degeneracy score, comprising:
receiving data comprising a representation of a nucleotide sequence, by one or more processors;
determining a window within the nucleotide sequence, by the one or more processors;
determining one or more amino acids corresponding to one or more codons in the window, by the one or more processors;
determining, by the one or more processors, one or more degeneracy values for the one or more amino acids in the window; and
combining the one or more degeneracy values in the window to determine the genetic degeneracy score, by the one or more processors.

2. The method of claim 1, further comprising sliding the window by at least one nucleotide across the nucleotide sequence, to generate a plurality of genetic degeneracy scores.

3. The method of claim 2, further comprising combining the plurality of genetic degeneracy scores into a final genetic degeneracy score.

4. The method of claim 1, further comprising identifying a conserved genetic sequence from a sample from a subject based at least in part on the genetic degeneracy score.

5. The method of claim 1, further comprising detecting a pathogen based at least in part on the identified conserved genetic sequence.

6. The method of claim 5, wherein the pathogen is an engineered pathogen.

7. The method of claim 1, further comprising determining a diagnosis of a disease in the subject or a prognosis of the disease in the subject based at least in part on the genetic degeneracy score.

8. The method of claim 4, wherein the sample is a cancer sample.

9. The method of claim 1, wherein further comprising designing primers based on the genetic degeneracy score.

10. The method of claim 1, further comprising selecting a biomarker based on the genetic degeneracy score.

11. The method of claim 1, wherein the nucleotide sequence comprises an engineered nucleotide sequence or a predicted nucleotide sequence.

12. The method of claim 4, wherein the nucleotide sequence is based on the sample from the subject.

13. The method of claim 4, wherein a genetic code for the subject is an artificial genetic code.

14. The method of claim 1, wherein the method is iterative with respect to the determining the window, the determining the one or more amino acids, the determining the one or more degeneracy values, or the combining the one or more degeneracy values.

15. The method of claim 2, wherein the method is iterative with respect to the sliding the window.

16. The method of claim 14, wherein the iterative method stops after a predetermined number of iterations.

17. The method of claim 14, wherein the iterative method stops after the sliding the window comprises sliding across the nucleotide sequence in its entirety.

18. The method of claim 1, wherein a first length of the window for a first iteration of the method can overlap with a second length of the window for a second iteration of the method.

19. The method of claim 1, further comprising using a codon look-up table for the determining the one or more amino acids corresponding to the one or more codons in the window or the determining the one or more degeneracy values.

20. The method of claim 1, wherein a length of the window in nucleotides is divisible by a length of a codon from the one or more codons, in nucleotides.

21. The method of claim 1, wherein the length of the window in nucleotides is divisible by three.

22. The method of claim 1, further comprising multiplying together the one or more degeneracy values in the window for the combining.

23. The method of claim 1, wherein the degeneracy value is a computed integer or a computed float.

24. The method of claim 1, further comprising combining the genetic degeneracy score with a determining a molecular clock rate for the nucleotide sequence.


SEQUENCE LISTING (1 sequence)

SEQ ID NO: 1 - DNA, 978 bp, synthetic construct
  source 1..978: mol_type = other DNA
atgaccaagagagtccggctcagtgactccttcaaccctgtctacccctatgaagatgaaagcacctcccaacacccctttataaacccagggtttatttccccaaatggcttcacccaaagcccagacggagttcttactttaaaatgtttaaccccgctaacaaccacaggcgggtctctacagctaaaagtgggagggggacttacagtagatgacactgatgggaccttacaagaaaacataggtaccaccacaccacttgttaagactgggcactctataggtttatccctaggagccggattgggaacagatgaaaataaactttgtaccaaattgggaaaaggacttacattcaattcaaacaacatttgcattgatgacaatattaacaccctgtggacaggaattaaccccaccgaagccaactgtcaaatgatggactccagtgaatctaatgattgcaaattaattctaacactagttaaaactggagccctagtcactgcatttgtttatgttataggagtatctaacaattttaatatgctaactacatacagaaatataaattttactgcggagctgttttttgattctgcgggtaatttactaactagcctgtcatccctaaaaactccacttaatcataaatcaggacaaaacatggctactggtgccattactaatgctaaaagtttcatgcccagcacaactgcttatcctttcaataataattctagagaaaaagaaaactacatttacggaacctgtcactacacagctagtgatcacactgcttttcccattgacatatctgtcatgcttaaccaaagagcaataagagctgatacatcatattgtattcgtataacttggtcctggaacacaggagatgccccagaggggcaaacctctgctacaaccctagttacctccccatttaccttttactacatcagagaagacgactga